(19) United States
(12) Patent Application Publication
(10) Pub. No.: US 2016/0206295 A1
(43) Pub. Date: Jul. 21, 2016

(54) APPARATUSES FOR ENDOSCOPIC CRYO-BIOPSY AND METHODS OF USE

(71) Applicant: Mordechai KRAMER, Petach-Tikva (IL)

(72) Inventors: Mordechai KRAMER, Petach-Tikva (IL); Roni ZVULONI, Haifa (IL); Paul KLEINBERGER, Jerusalem (IL)

(73) Assignee: Mordechai Kramer, Petach-Tikva (IL)

(21) Appl. No.: 14/904,441

(22) PCT Filed: Jul. 10, 2014
(86) PCT No.: PCT/IL2014/050627

§ 371 Date: Jan. 12, 2016

Related U.S. Application Data
(60) Provisional application No. 61/845,375, filed on Jul. 12, 2013.

(51) Int. Cl. A61B 10/02

(52) U.S. Cl. CPC A61B 10/0275

(57) ABSTRACT

An endoscopic cryoprobe system, comprising: a low friction canula slidable within an instrument channel of an endoscope tube; and, a low friction cryoprobe slidable within the canula and provided with a tissue specimen acquiring tip at the distal end of the cryoprobe, where the canula is sized and shaped to nest the cryoprobe and the specimen within for protection during retraction.

### RELATED APPLICATION/S

This application claims priority from U.S. Provisional Patent Application No. 61/845,375 filed 12 Jul. 2013. The contents of the above application are incorporated by reference as if fully set forth herein.

### FIELD AND BACKGROUND OF THE INVENTION

The present invention, in some embodiments thereof, relates to biopsy methods and equipment and, more particularly, but not exclusively, to apparatuses and methods for performing endoscopic cryo-biopsy.

In currently available systems for cryo-biopsy, in a technique similar to the technique used to perform classical forceps transbronchial biopsies, the cryoprobe is introduced through a flexible bronchoscope instrument channel (often called a “work channel”) under fluoroscopic control, and is introduced into peripheric lung areas previously selected according to CT findings. Cold is applied for few seconds, causing tissue touching the probe tip of the cryoprobe to freeze and adhere to the tip, and then the cryoprobe with the frozen tissue sample adhering to the tip is removed, along with the bronchoscope. Samples are then removed from the cryoprobe tip and are sent to pathology in formaldehyde solution for analysis.

Repeat biopsies using these current techniques require re-insertion of the bronchoscope into the patient's body, re-navigation of the bronchoscope to the relevant area of interest in the body with associated cost in inconvenience, physician time, added danger of complications, and increased patient discomfort or extended time of anesthesia.

The literature regarding transbronchial lung biopsy using cryoprobes presents the use of cryoprobes that have a rounded tip that is pushed and inserted into tissue. The use of these types of cryoprobe tips is associated with significant variability in the size of the specimen. Specimens that are too large may cause excess bleeding. Specimens that are too small may not provide sufficient tissue for reliable histopathology. Specimens of ˜10 mm² are accepted as the preferred size with enough material to achieve conclusive histopathology results, however using current tools and methodologies there is no guarantee of sampling the accepted specimen size.

Some references which describe tools and methods in this field are: U.S. Pat. No. 3,598,108; U.S. Pat. No. 3,630,192; U.S. Pat. No. 5,452,584; No. U.S. Pat. No. 5,494,039; U.S. Pat. No. 6,540,694; U.S. Pat. No. 6,551,255; U.S. Pat. No. 7,311,672; U.S. Pat. No. 7,611,475; U.S. Pat. No. 8,216,153; U.S. Pat. No. 8,303,517; U.S. Pat. No. 8,308,718; U.S. Pub. No. 2003/0208135; U.S. Pub. No. 2008/0286800; U.S. Pub. No. 2009/0019877; U.S. Pub. No. 2010/0016847; U.S. Pub. No. 2011/0071427; U.S. Pub. No. 2012/0071868, the disclosures of which are incorporated herein in their entireties.

Additional background art includes: Virginia Pajares et al., “Transbronchial Lung Biopsy Using Cryoprobes”, Arch Bronconeumol. 2010;46(3):111-115; A. Gil de Bernabê, et al, “Transbronchial lung biopsy with cryoprobes under outpatient regime”, Ambulatory Anaesthesia, 2AP3-4; and, Oren Fruchter, Ludmila Fridel, Dror Rosengarten, Yael Raviv, Viktoria Rosanov, Mordechai R. Kramer, “Transbronchial cryo-biopsy in lung transplantation patients: First report”, Respirology. 2013 May, 18(4):669-73, doi: 10.1111/resp.12037, the disclosures of which are incorporated herein in their entireties.

### SUMMARY OF THE INVENTION

There is provided in accordance with an exemplary embodiment of the invention, an endoscopic cryoprobe system, comprising: a low friction canula slidable within an instrument channel of an endoscope tube; and, a low friction cryoprobe slidable within the canula and provided with a tissue specimen acquiring tip at the distal end of the cryoprobe, where the canula is sized and shaped to nest the cryoprobe and the specimen within for protection during retraction.

In an embodiment of the invention, the specimen is retained by at least one of the canula or cryoprobe during retraction by at least one of a compression fit, frozen adhesion, adhesion to a hydrophilic material or by being speared by the tip.

In an embodiment of the invention, the tip comprising sharpened edges at the leading edge of the tip and a hollow cavity extending from the leading edge towards the proximal end of the tip.

In an embodiment of the invention, the hollow cavity is symmetrical around a longitudinal axis of the cryoprobe.

In an embodiment of the invention, the hollow cavity is shaped like a cone, with the base of the cone at the leading edge.

In an embodiment of the invention, the hollow cavity is shaped like a cylinder.

In an embodiment of the invention, the canula comprising temperature control circuitry including at least one of switching polarity conductive wiring or resistive heating conductive wiring, and capable of providing at least one of heating or cooling to selected surfaces of the canula. In an embodiment of the invention, selected surfaces are at least a portion of at least one an inner surface of the canula, an outer surface of the canula, an outer surface of the cryoprobe, or an inner surface of the cryoprobe. In an embodiment of the invention, the sensor is located in the cryoprobe.

In an embodiment of the invention, the tip configured to be radially expandable around a longitudinal axis of the cryoprobe. In an embodiment of the invention, the tip is configured with radially expanding cooling elements to make the tip expandable.

In an embodiment of the invention, the canula is configured with a plurality of leaves located on the distal end of the canula to radially expand around a longitudinal axis of the canula.

In an embodiment of the invention, at least a portion of at least one of the cryoprobe or the canula is coated with a hydrophobic coating to enhance adhesion.

In an embodiment of the invention, at least a portion of at least one of the cryoprobe or the canula is coated with a hydrophilic coating to reduce adhesion.

In an embodiment of the invention, an inner surface of the cavity is cooled to enhance adhesion of the specimen thereto.

There is provided in accordance with an exemplary embodiment of the invention, an endoscopic cryoprobe system, comprising a temperature controlled canula that has at least one heated or cooled surface.

In an embodiment of the invention, the at least one heated or cooled surface is at least one of an inside surface of the canula or outside surface of the canula.

In an embodiment of the invention, the temperature is controlled by conductive wires embedded within the canula or located on the surface of the canula.

In an embodiment of the invention, the system further comprises a sensor and a temperature controller where the controller is programmed to change the heating or cooling and the degree of heating or cooling to the canula in response to feedback from the sensor. In an embodiment of the invention, the sensor is located near the distal end of the canula.

There is provided in accordance with an exemplary embodiment of the invention, an endoscope cryoprobe system, comprising a component with an expanding tip.

In an embodiment of the invention, the component is an expanding distal end of a canula is comprised of radially expanding leaves.

In an embodiment of the invention, the component is an expanding cryoprobe distal end comprised of flexible cooling elements.

There is provided in accordance with an exemplary embodiment of the invention, A method for performing an endoscopic cryoprobe biopsy, comprising: cooling a tip of a cryoprobe in contact with body tissue, causing the tissue to be sampled to adhere to the tip through freezing; pulling the cryoprobe tip along with a torn off tissue specimen back into a canula; pulling the canula out of an endoscope tube inserted into the body lumen with the cryoprobe and the specimen nested within for protection; and, removing the specimen from the canula for storage.

In an embodiment of the invention, the tip is a shaped tip and further comprising pushing the shaped tip with a hollow cavity into the tissue to be sampled, thereby forcing and adhering the tissue into the cavity, prior to pulling the cryoprobe tip back into the canula.

In an embodiment of the invention, the method further comprises automatically tearing off the tissue specimen using an automatic tearing mechanism activated by a spring loaded mechanism to increase consistency of the sampling.

In an embodiment of the invention, the method further comprises activating the automatic tearing mechanism by using a physician operated control.

In an embodiment of the invention, the method further comprises using a timer to set the timing of at least one of the retraction of the automatic tearing mechanism or the application of temperature control to the cryoprobe.

In an embodiment of the invention, the timer activates retraction of the cryoprobe after a timer controlled application of the cooling.

In an embodiment of the invention, the specimen is pulled back into an expandable cryoprobe tip thereby retrieving a specimen larger in diameter than at least one of the cryoprobe or canula.

In an embodiment of the invention, the method further comprises compressing the specimen by pulling the cryoprobe tip along with a torn off tissue specimen back into the canula.

In an embodiment of the invention, the method further comprises encouraging adhesion of the specimen to at least a portion of the system by performing at least one of cooling the portion of the system or coating the portion with an adhesion facilitating coating. In an embodiment of the invention, the at least a portion is at least one of the cryoprobe tip, a hollow cavity at the distal end of the cryoprobe tip or the inner surface of the canula.

In an embodiment of the invention, the method further comprises reinserting the canula and the cryoprobe into the endoscope tube to take an additional sample.

In an embodiment of the invention, the method further comprises discouraging adhesion of the specimen to at least a portion of the system by performing at least one of heating the portion of the system.

There is provided in accordance with an exemplary embodiment of the invention, A method for acquiring consistent size and shape biopsy specimens, comprising:

pushing a cryoprobe tip with a specially shaped hollow cavity into tissue to be sampled;

forcing a tissue specimen into the cavity to fill the cavity by the pushing; adhering the tissue specimen in the cavity; and, retracting the specimen using at least one of a cryoprobe or canula.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example, are not necessarily to scale and are for purposes of illustrative discussion of embodiments of the invention. In this regard, the description taken with the drawings makes apparent to those skilled in the art how embodiments of the invention may be practiced.

In the drawings:

FIG. 1 is a schematic view of a prior art endoscopic system;

FIG. 2A is a more detailed view of the prior art endoscopic system of FIG. 1;

FIG. 2B is a cross-sectional view along the minor axis of the prior art endoscopic system of FIG. 2A;

FIG. 2C is cross-sectional view along the major axis of the prior art endoscopic system of FIG. 2A;

FIGS. 3A-3D are partially cross-sectioned views of an endoscopic cryoprobe system for retrieving tissue samples, in accordance with an exemplary embodiments of the invention;

FIG. 3E is a flowchart showing a method of using the endoscope cryoprobe system depicted in FIGS. 3A-3D, in accordance with an exemplary embodiment of the invention;

FIG. 4A is a schematic view of a thermoelectric heating and/or cooling endoscopic cryoprobe system, in accordance with an exemplary embodiment of the invention;

FIG. 4B is a schematic view of a resistive heating endoscopic cryoprobe system, in accordance with an exemplary embodiment of the invention;

FIG. 4C is a flowchart showing a method of using the endoscope cryoprobe system depicted in FIGS. 4A-4, in accordance with an exemplary embodiment of the invention;

FIG. 5A is a schematic view of the performance of a prior art cryoprobe system;

FIG. 5B is a schematic view of an angled, concave-shaped tip endoscopic cryoprobe system and its performance, in accordance with an exemplary embodiment of the invention;

FIG. 5C is a schematic view of a cylindrically, concave-shaped tip endoscopic cryoprobe system and its performance, in accordance with an exemplary embodiment of the invention;

FIG. 5D is a schematic view of a partially insulated, angled, concave-shaped tip endoscopic cryoprobe system and its performance, in accordance with an exemplary embodiment of the invention;

FIG. 6 is a schematic view of a side harvesting endoscopic cryoprobe system, in accordance with an exemplary embodiment of the invention;

FIG. 7A is a schematic view of an endoscopic cryoprobe system with a kink resistant canula, in accordance with an exemplary embodiment of the invention;

FIG. 7B is a more detailed view of the canula of the endoscopic cryoprobe system of FIG. 7A, in accordance with an exemplary embodiment of the invention;

FIG. 7C is a more detailed view of an endoscopic cryoprobe system with a kink resistant canula and a transparent tip, in accordance with an exemplary embodiment of the invention;

FIGS. 8A-8B are schematic views of an automatic tearing mechanism, in accordance with an exemplary embodiment of the invention;

FIG. 8C is a flowchart describing a method of using the mechanism of FIGS. 8A-8B, in accordance with an exemplary embodiment of the invention;

FIG. 9A is a schematic view of an endoscopic cryoprobe system with a coolant source, in accordance with an exemplary embodiment of the invention;

FIG. 9B is a detailed schematic view of the endoscopic cryoprobe system of FIG. 9A, in accordance with an exemplary embodiment of the invention;

FIG. 9C is a cross-sectional view of the system of FIG. 9B, in accordance with an exemplary embodiment of the invention;

FIG. 10A is a schematic view of an endoscopic cryoprobe system with a sliding internal element, in accordance with an exemplary embodiment of the invention;

FIGS. 10B-10C are schematic views showing a sampling procedure using the endoscopic cryoprobe system of FIG. 10A, in accordance with an exemplary embodiment of the invention;

FIG. 10D is a schematic view of an endoscopic cryoprobe system with a sliding and expanding internal element, in accordance with an exemplary embodiment of the invention;

FIG. 10E is a flowchart of a method of use for the systems depicted in FIGS. 10A-10D, in accordance with an exemplary embodiment of the invention;

FIGS. 11A-11D are a graphical depiction of a sampling procedure using a endoscopic cryoprobe system with an expanding leaf canula, in accordance with an exemplary embodiment of the invention;

FIG. 11E is a flowchart of a method of use for the systems depicted in FIGS. 11A-11D, in accordance with an exemplary embodiment of the invention;

FIGS. 12A-12B are a perspective view of the tip of the expanding leaf canula of FIGS. 11A-11D in more detail, in accordance with an exemplary embodiment of the invention; and,

FIG. 13A-13C are a schematic view of an endoscopic cryoprobe system with a reduced profile cryoprobe tip, in accordance with an exemplary embodiment of the invention.

### DESCRIPTION OF SPECIFIC EMBODIMENTS OF THE INVENTION

The present invention, in some embodiments thereof, relates to biopsy methods and equipment and, more particularly, but not exclusively, to apparatuses and methods for performing endoscopic cryo-biopsy.

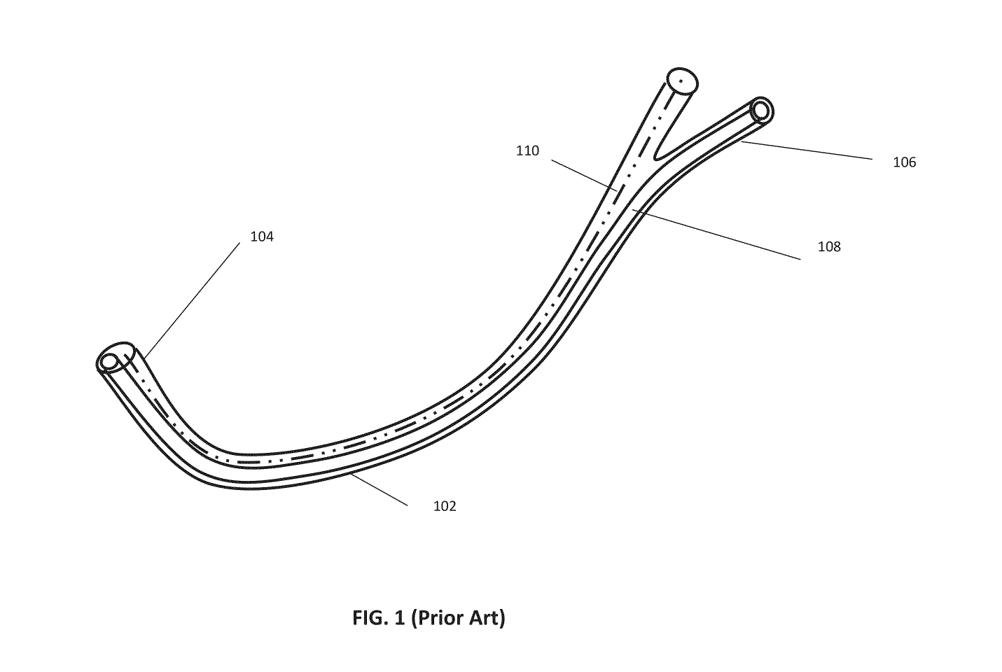

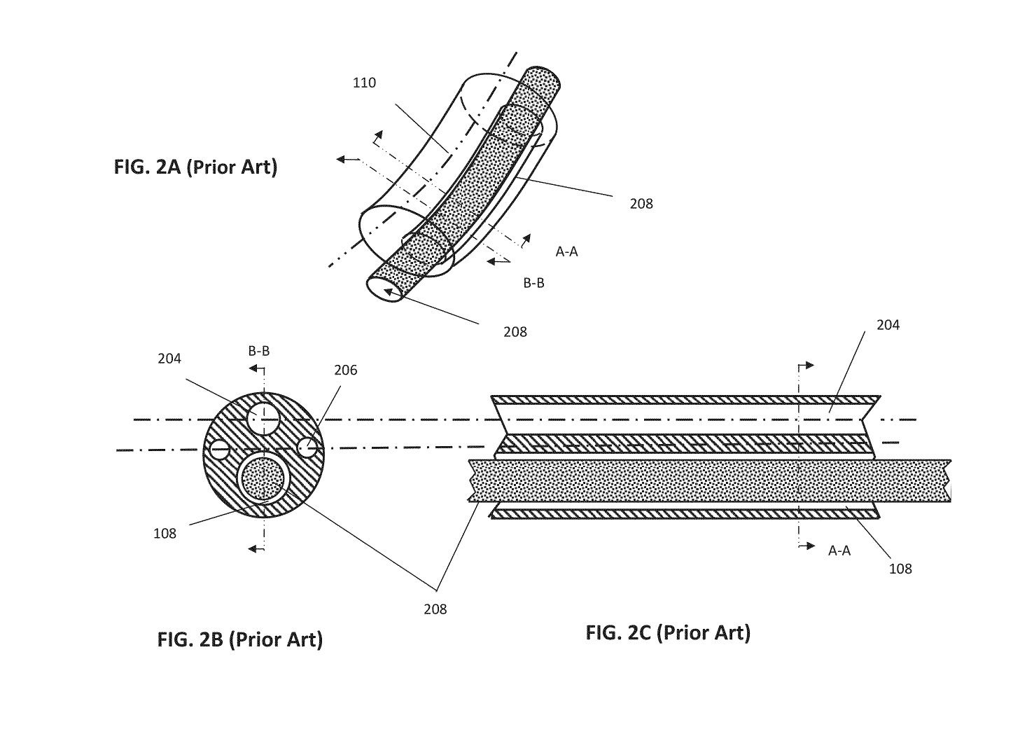

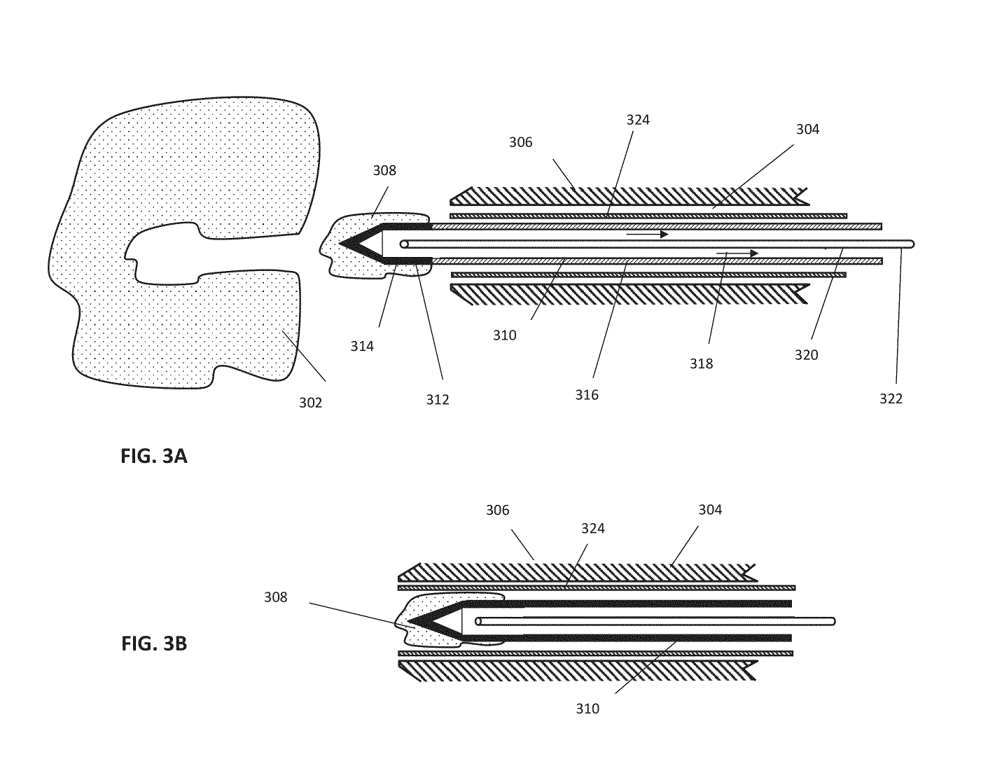

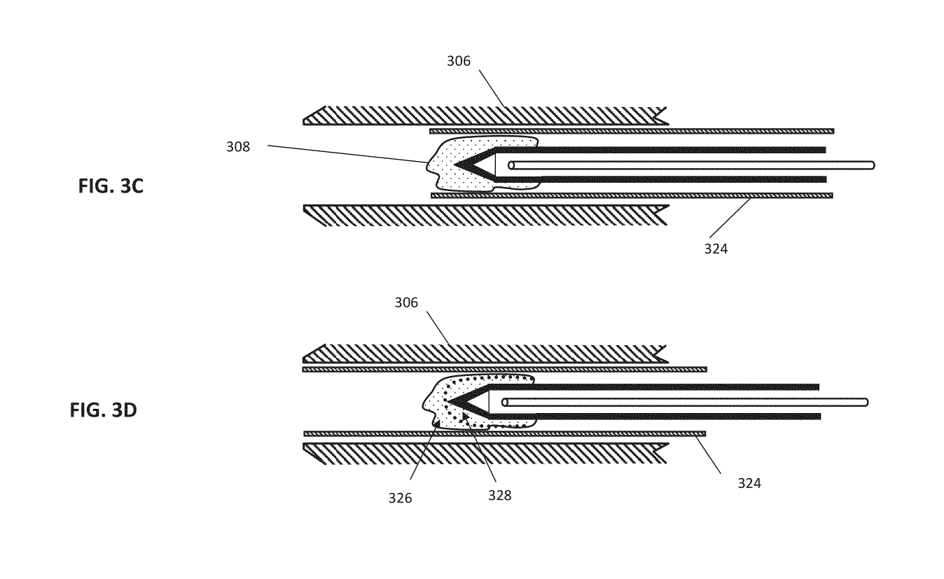

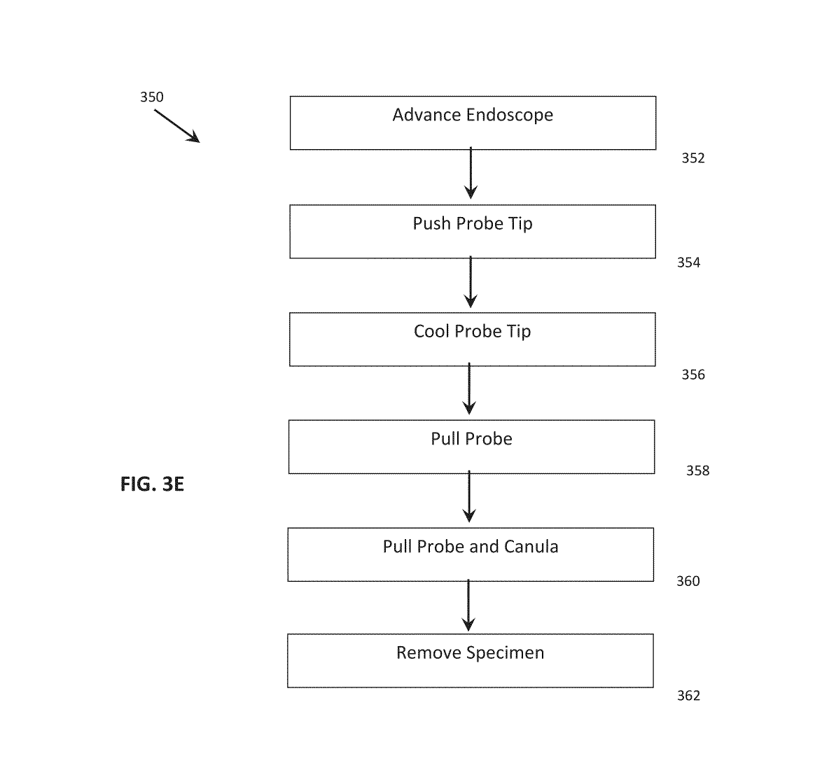

For purposes of better understanding some embodiments of the present invention, as illustrated in FIGS. 3A-4C and FIGS. 5B-12B of the drawings, reference is first made to the construction and operation of a prior art endoscopic system as illustrated in FIGS. 1 and 2A-2C.

FIG. 1 shows a typical endoscope system 102 used to examine the interior of a hollow organ or cavity of the body. Endoscopes typically include a scope insertion tube with distal (penetrating) head or tip 104, for insertion in a body, and also include a proximal end 106 that remains outside the body. The insertion tube typically includes instrument channel 108, light transmission channel(s), imaging channels (for example via optical fibers or signal lines in case of CCD embedded at the endoscope distal tip), and one or more maneuvering channels for the maneuvering rods that mechanically control the bending of the distal portion of the insertion tube. For simplicity, all these subsystems are symbolically represented in the figure by a thick dotted line 110 showing no details of the subsystems. The proximal end 106 typically includes a connection to light source, means for capturing and presenting the images, maneuvering means, and the entrance to the instrument channel 108. Endoscopes further often include means for injection of liquids (e.g. saline) and means for suction to rinse the working areas, which are often within natural conduits of the body. These means are also not shown.

FIG. 2A is a more detailed, partial, perspective view of the prior art endoscopic system 102 of FIG. 1. Shown within the endoscope's instrument channel 108 is an instrument 208, for example forceps.

FIG. 2B is a cross-sectional view along the minor (transverse) axis of the prior art endoscopic system of FIG. 2A. Cross-section A-A, shown in FIG. 2C, shows typical imaging 204 and illuminating 206 channels along with an instrument 208, such as a forceps, in the instrument channel 108.

FIG. 2C is cross-sectional view along the major (longitudinal) axis of the prior art endoscopic system of FIG. 2A. Cross-section B-B, shown in FIG. 2B, depicts the instrument 208 in the instrument channel 108. Typical dimensions for the instrument channel diameter are 1-5 mm.

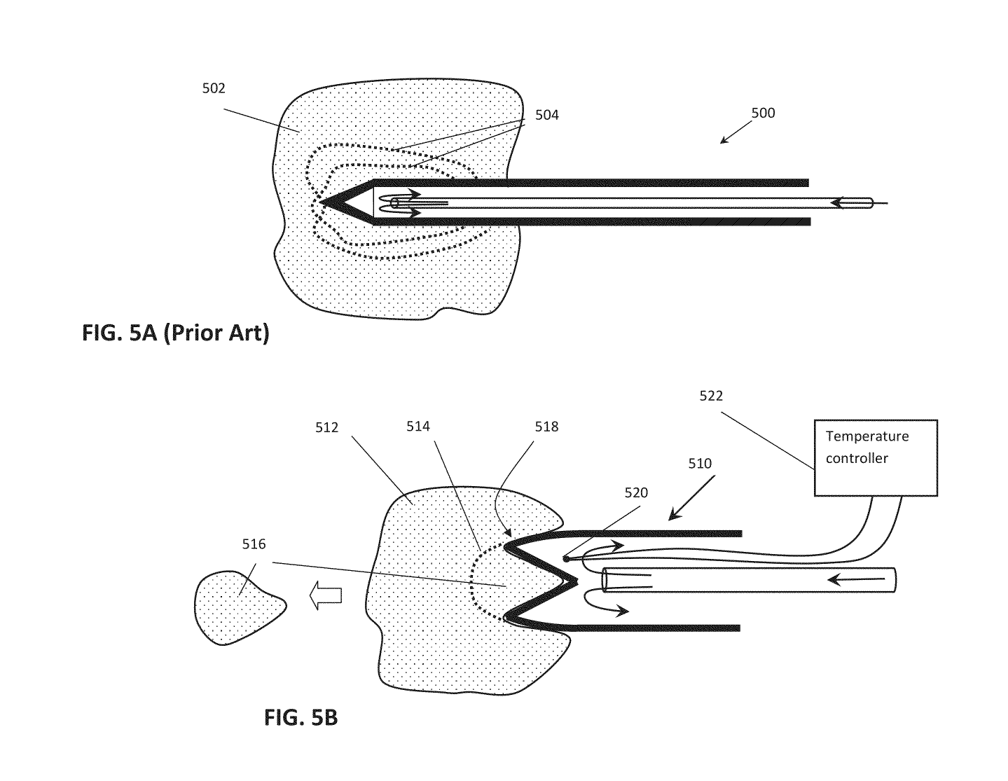

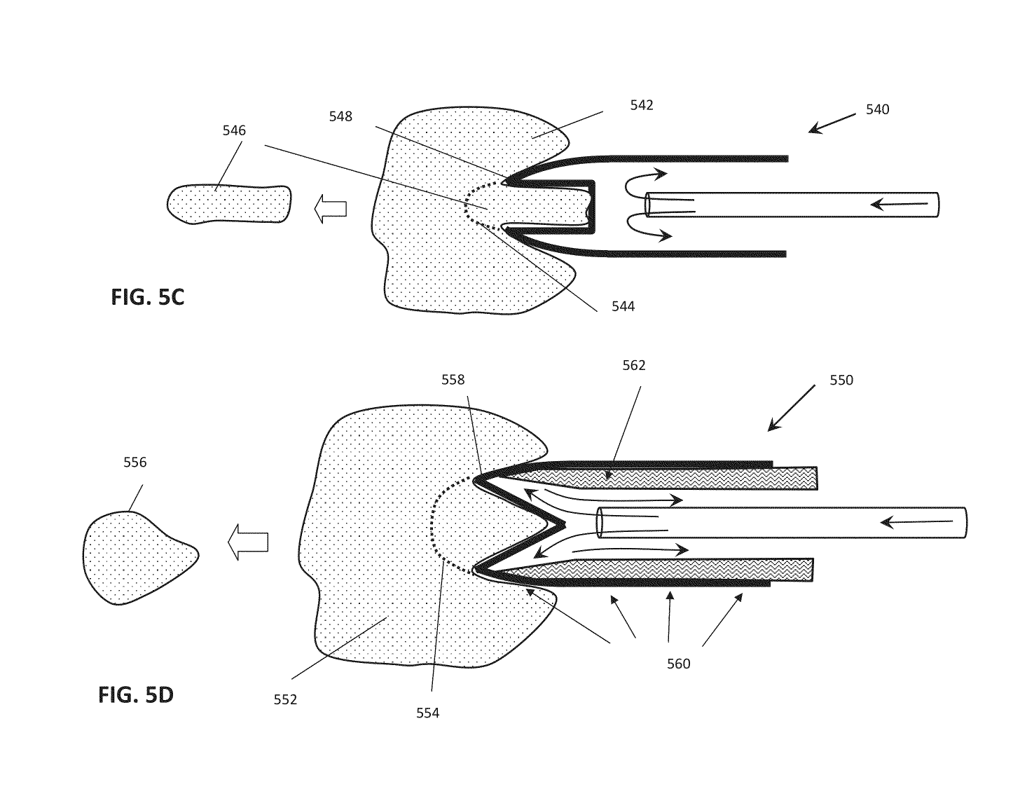

A prior art cryoprobe tip configuration is shown in FIG. 5A, described in more detail below.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Generally, exemplary embodiments of the invention describe endoscope systems which allow for specimens/samples to be taken from the body without removing the whole endoscope assembly, for example by sliding out the canula and/or the cryoprobe together (with the cryoprobe and specimen nested within the canula) and leaving in at least the outer endoscope tube. Some exemplary embodiments describe specially constructed endoscope system components (e.g. canula, cryoprobe, endoscope instrument channel) which are configured to reduce friction between the moving parts of the system. Other exemplary embodiments of the invention describe endoscope cryoprobe systems which allow for varying forms of temperature control, for example being able to heat and/or cool the surface, or a portion of the surface, of the cryoprobe to either enhance and/or dissuade adhesion of body tissue to portions of the cryoprobe and/or canula. In some exemplary embodiments of the invention, the cryoprobe exhibits a specially shaped tip with a hollow cavity (which is filled by the tissue specimen during collection) to ensure a particular specimen size and/or shape. In some embodiments of the invention, adhesion is encouraged inside the cavity in the tip, and adhesion is discouraged outside the cavity. Optionally, the tip is expandable to a diameter larger than the diameter of the canula, for example to retrieve a sample size that is larger than the diameter of the canula but which is compressed upon withdrawal from the body. In some embodiments of the invention, the specimen is retained on the cryoprobe tip by at least one of a compression fit, frozen adhesion, adhesion to a hydrophilic material or by being speared by the sharp tip.

An aspect of some embodiments of the invention relates to a user to collecting multiple specimens from the patient's body without having to remove the endoscope from the patient using specially configured endoscopic tools. In an embodiment of the invention, a canula and/or the cryoprobe are retracted from the body while the endoscope tube stays in situ. In some embodiments of the invention, movement of the canula and/or cryoprobe is facilitated by providing composite and/or multilayered construction to at least a portion of the canula and/or the cryoprobe, for example by applying low friction, hydrophobic and/or hydrophilic materials to them. In some embodiments of the invention, movement of the canula and/or cryoprobe is facilitated by providing selective temperature control, including heating and/or cooling, to at least one of the canula and the cryoprobe. In some embodiments of the invention, movement of the canula and/or cryoprobe is facilitated by providing a sliding insert to at least one of the canula and cryoprobe.

An aspect of some embodiments of the invention relates to a user to collecting a more consistently sized and/or larger and/or more consistently shaped specimen during a biopsy procedure. In some embodiments of the invention, the cryoprobe is provided with a specially shaped and/or configured tip which is designed to force the specimen into a hollow cavity of a predetermined size and/or shape located at the distal end of the tip (where distal is farthest into the body, and proximal is the closest to the attending physician outside the body). In some embodiments of the invention, the tip is provided with sharp and/or cutting edges to instigate desired tissue separation or tearing.

In some embodiments of the invention, the tip is provided with the ability to expand, to accommodate a specimen that is larger in diameter than the diameter of the probe and/or canula. In some embodiments of the invention, expanding and/or flexible cooling elements are provided to the cryoprobe which expand when pushed out of the cryoprobe to tear a specimen of tissue of that is larger in diameter than the diameter of the probe and/or canula. In some embodiments of the invention, expandable and/or flexible cooling elements are provided to a sliding insert to accommodate the larger specimen.

In some embodiments of the invention, expanding and/or flexible leaves are provided around the distal circumference of the canula, where the plurality of leaves expand radially from the longitudinal axis of the canula when a specimen larger than the diameter of the canula is pulled into the lumen of the canula.

An aspect of some embodiments of the invention relates to an automatic tearing mechanism which is provided to an endoscopic cryoprobe system to provide more consistency and/or reproducibility to tissue sampling during biopsies. In an embodiment of the invention, a spring actuated assembly is provided to the mechanism, whereby the spring is operated by pushing or pulling at least one of a knob, a pin, and a lever, as examples. Other known methods include electrical actuators, pneumatic actuators, electrical motors, electromagnetic actuators etc. In some embodiments of the invention, the mechanism is at least partially automated by a timer. Optionally, the timer controls at least one of cooling and triggering retraction.

It should be understood that the term “endoscope” as used herein includes cystoscopes, bronchoscopes, gastroscopes, neuroendoscopes, and all similar endoscopic tools. The apparatuses and methods described herein are used, in some embodiments of the invention, for cryo-biopsy procedures such as transbronchial lung biopsies and other biopsies.

Referring now to the drawings, FIGS. 3A-3D are partially cross-sectioned views of an endoscopic cryoprobe for retrieving tissue samples enabling safe removal of the biopsy specimen 308 without the need for removal and re-insertion of the whole endoscope 306, in accordance with an exemplary embodiments of the invention. In an embodiment of the invention, a flexible canula 324 is provided which is used to allow the sliding of the canula 324 in and out of the endoscope 306 without having to remove the whole endoscope 306 and/or while also protecting the specimen 308 during withdrawal. The procedure/method 350 is described in more detail below and in conjunction with FIG. 3E.

A flexible cryoprobe 310 (or “probe”), optionally with 1.5 mm-3.0 mm outer diameter, is shown inserted in an instrument channel 304 of an endoscope 306 inserted in a body. (To simplify the drawing, lighting, imaging, and maneuvering channels etc. are not shown in the Figures.) The probe 310 has a cryotip 312, made of heat-conducting material (such as stainless steel) with a sealed connection to a flexible tube 316 (typically a plastic tube such as PTFE, FEP, etc.) at the rear side of the tip. The flexible tube contains a thin pressure tube 322 (optionally metal) which is used to bring high pressure gas to the cryotip 312. These small diameter metal tubes, which may have an outer diameter of 0.5-0.8 mm, are flexible enough to not inhibit endoscope maneuvering. High pressure incoming cooling gas (for example CO2, N2O, Argon) flows through the thin, high pressure tube 322 up to an orifice 314 at its distal end where the gas expands and flows out 318 between the pressure tube 322 and the flexible tube 316 and is released to the atmosphere near the endoscope 306 proximal end. Gas expansion as the gas passes the orifice 314 reduces the gas temperature to low (sub-zero) temperatures because of the Joule-Thomson effect. The low temperature gas cools the probe tip.

The flexible canula 324 is configured to allow for smooth insertion of the canula into the endoscope instrument channel and/or for smooth insertion of the cryoprobe 310 into the canula 324.

The probe 310 and canula 324 are inserted into the endoscope 306 and the endoscope 306 is further advanced (352) into patient body through a natural body lumen. Alternatively, the canula 324 and cryoprobe 310 can be advanced into the endoscope 306 after the endoscope has been advanced (352). Once in position near an intended biopsy site, the probe tip 312 is pushed (354) forward so that it extends out of the canula 324 into the tissue 302, and the probe tip 312 is cooled (356). This makes tissue stick to the probe tip 312. This is shown in an exemplary embodiment in FIG. 3A. The probe 310 is then pulled (358) back from the tissue 302, which detaches the frozen tissue from its place in the body, creating the sampled specimen 308.

FIG. 3B shows the probe 310 and specimen 308 pulled (358) back by the cryoprobe 310 and into the canula 324.

FIG. 3C shows the probe 310 and canula 324 together being pulled (360) out of the body with the specimen 308 ‘nested’ inside the canula 324. By this method the specimen 308 is protected from frictional contact that might damage it or cause it to separate from the probe 310 while being removed from the endoscope 306. While canula 324, probe 310, and specimen 308 are being pulled out of the body through the endoscope instrument channel, the endoscope 306 remains in place in the body. Once the probe 310 and the canula 324 are outside the endoscope 306, the specimen 308 can be removed (362) from the probe tip 312 and preserved in a vial for storage and for histopathology analyses, and the canula 324+cryoprobe 310 can be re-inserted into the endoscope 306 to take more samples.

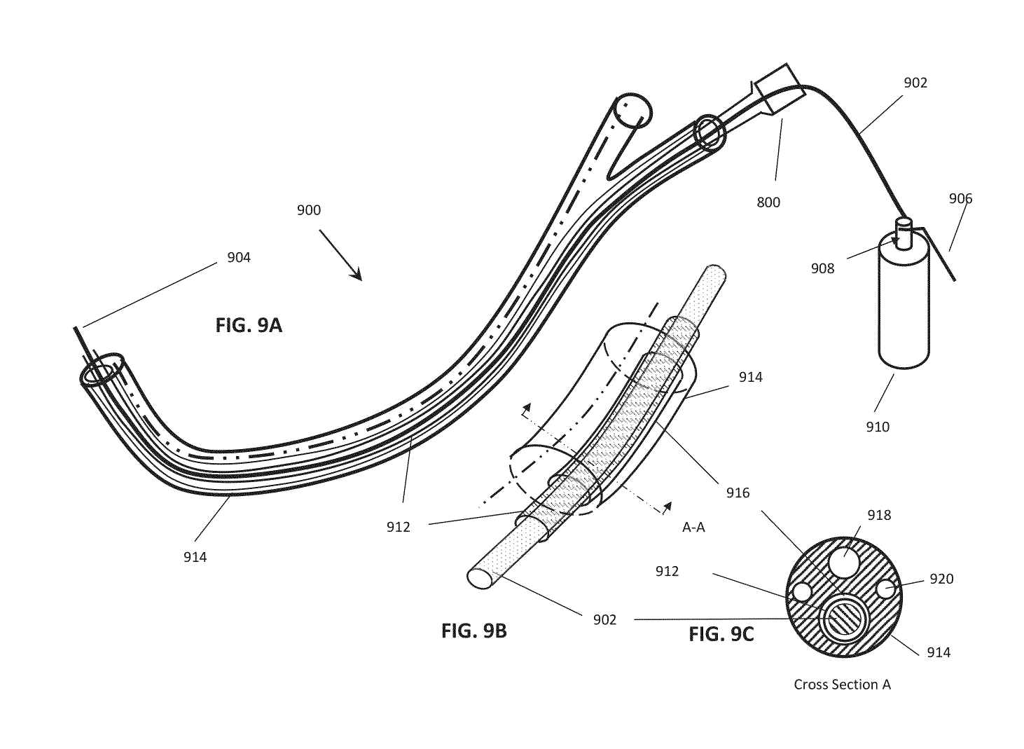

FIG. 3D discloses another technique for removal of the specimen 308, in an exemplary embodiment of the invention. Using this technique the cryoprobe 310 continues to cool the specimen 308 but with somewhat less cooling power than was required for harvesting the specimen 308. At this lower cooling temperature, the near tissue 328 of the specimen 308 adheres to the cryoprobe 310 via the sub-zero temperatures at the probe-specimen interface, however, the cooling is controlled (see for example the discussion with respect to FIG. 9) so as to not be so cold so that the temperature of the far tissue 326 is slightly higher, and warm enough so that this portion of the specimen 308 does not adhere/freeze to the canula 324. Under these conditions the probe 310 and specimen 308 combination can be removed safely through the canula 324 without sticking to the canula 324.

The partial-cooling technique just described with respect to FIG. 3D can also be used in a system without canula 324, in an embodiment of the invention. With this system and method the specimen 308 continuously adheres to the cryoprobe 310 while the cryoprobe 310 itself is removed out of the endoscope 306, with limited continuing cooling of the probe 310 and/or a non-stick coating on the walls of the endoscope instrument channel, the specimen 308 can remain stuck to the probe 310 while not sticking to the instrument channel of the endoscope 306. Once the specimen 308 is out of the body, cooling is stopped and the specimen 308 is moved to a standard vial for preservation and pathology analysis.

As an option, embodiments of the present invention use a non-stick coating (e.g. a hydrophilic coating, for example, Hydromer® by Hydromer, Inc., or HYDAK® by Biocoat, Inc.) on one or more surfaces of the canula 324. A hydrophilic coating on the internal surface that touches the specimen 308 will facilitate sliding the (non-frozen (far tissue 326) exterior portion of the specimen 308 along the canula 324 to remove it from the body while leaving endoscope 306 and canula 324 in place. Alternatively, the endoscope working channel may be provided with such a coating and the removing of the specimen 308 (with non-frozen exterior 326) can be practiced without canula 324, with the cryoprobe 310 inserted directly in the endoscope instrument channel. (This option can be practiced with or without coating, but it is anticipated that such a coating will facilitate the process.)

Providing a hydrophilic coating between canula 324 and the endoscope instrument channel (applied to either the exterior of the canula or the interior surface of the channel walls, or both) will facilitate movement of the canula 324 in and out of the endoscope 306.

In a further alternative option, the interior walls of canula 324 and/or endoscope 306 channel may be provided with a hydrophobic coating, which may facilitate passage of a specimen 308 whose exterior portions are also fully or partially frozen, or are unfrozen but approaching freezing temperatures (i.e. all or most of the sample being “near tissue”). In this case the hydrophobic layer serves to discourage adhesion of freezing or frozen or partially frozen tissue to the surrounding material (the interior walls of the canula 324 or the interior walls of the working channel 306 if no canula 324 is present), since the hydrophobic layer tends to reject contact with water, thereby discouraging freezing tissue from entering into adhesive contact with the surrounding surface.

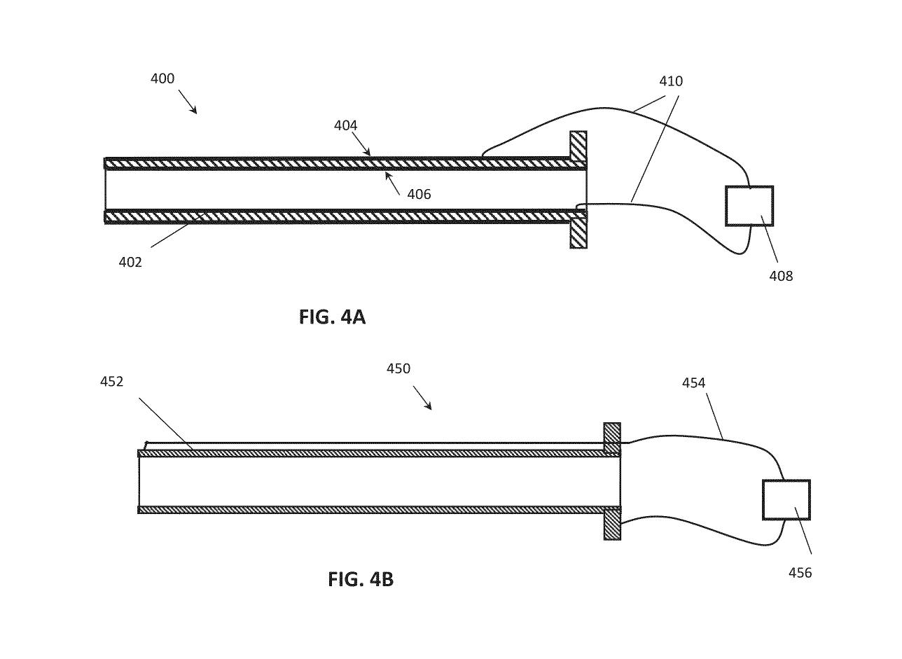

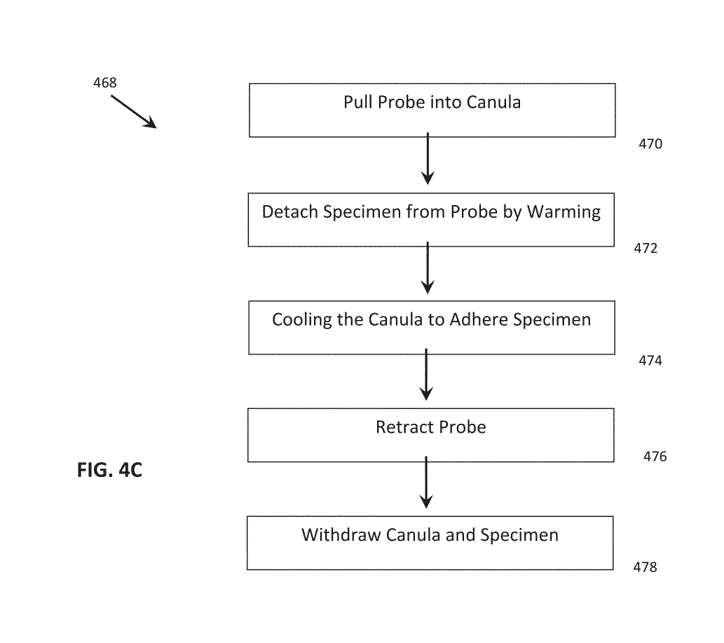

FIG. 4A is a schematic view of a thermoelectric heating and/or cooling endoscopic cryoprobe system 400 (only the canula 402 is shown of the system 400), in accordance with an exemplary embodiment of the invention. In an embodiment of the invention, heating and/or cooling are provided by the system 400 to prevent adhesion of a specimen to components of the system during movement. FIG. 4A shows schematically a canula 402 equipped for thermoelectric (Peltier) heating and/or cooling. In an embodiment of the invention, switching the polarity of the connections 410 to the electrical source 408 (e.g. DC source) switches heating and cooling between the outer surface 404 of the canula 402 and the inner surface 406 of the canula 402. Since the endoscope body (not shown) in which the canula 402 is inserted may function as a heat sink, the arrangement shown in FIG. 4A enables controlled heating and controlled cooling of the canula inner surface 406. Conductive lines as required for a Peltier system may be embedded in the canula 402 body.

FIG. 4B is a schematic view of a resistive heating endoscopic cryoprobe system 450, in accordance with an exemplary embodiment of the invention. In an exemplary embodiment of the invention, an electric source (AC or DC) 456 used for resistive heating of the canula 452. Here, too, conductive lines 454 can be embedded in the canula 452 or wrapped around it, for example in the form of longitudinal resistive lines, or in the form of a spiral in or around the canula walls.

Using Peltier cooling or other cooling and/or heating of the canula, it is possible to use a cryoprobe to harvest a tissue sample/specimen. In an embodiment of the invention as shown in the flowchart 468 of FIG. 4C, pulling (470) the probe into the canula, then heating the probe or simply allowing the probe temperature to rise enough through lack of cooling to enable tissue to detach (472) from the probe, while simultaneously or subsequently cooling (474) the canula enough to cause the tissue to adhere to the canula (instead of adhering to the probe). The probe can then be retracted (476), and the canula with the adhering specimen can be withdrawn (478) later. This technique helps to preserve the tissue at low temperature from right after harvest until its transition from the canula to a cooler for storage. Keeping the tissue at low temperature immediately from the moment it was harvested better preserves information that otherwise would not be available during analysis. This technique makes it unnecessary to cool the probe for a long period of time, making it possible to use a gas container which is both inexpensive and small and easy to manipulate, for example that which is shown and described with respect to FIG. 9A.

Additionally, alternatively and/or optionally, the canula can be heated (with Peltier heating or resistive heating or other method) to above-zero temperatures while a specimen adhering to a cryoprobe in the canula is being held to the probe by sub-zero temperatures of the probe. In this embodiment, the specimen, though cold, will not stick to the canula and can be safely pulled out as was described with respect to FIG. 3D.

FIG. 5A is a schematic view of the performance of a prior art cryoprobe 500. One problem in current methods of using cryoprobes to harvest biopsy tissue samples is that the amount of tissue harvested tends to be highly variable and unpredictable. FIG. 5A shows a cryoprobe with a conventional rounded tip as is used today. A probe tip like that in FIG. 5A creates an unpredictable tearing pattern 504 when tissue adhering to a cooled cryoprobe tears away from tissue 502 still connected to the body. The dotted lines in the figure show examples from among a variety of equally probable tearing lines (surfaces along which the tissue disconnects from the body) and show that the size and shape of the torn tissue is unpredictable.

FIGS. 5B-5D show specially shaped cryoprobe tips 510, 540, 550 which are used to reduce the variability of the size and/or shape of the specimens 516, 546, 556 collected, in some embodiments of the invention.

5B is a schematic view of an angled, concave-shaped tip 510 endoscopic cryoprobe and its expected performance, in accordance with an exemplary embodiment of the invention. In an embodiment of the invention, the leading edge 518 is sharp enough to cut tissue 512 into which the probe is advanced. This sharp structure 518 facilitates filling the concavity with a sampled tissue specimen 516. This structure tends to cause the tissue to tear along a surface 514 which meets these pointed structures 518, yielding a predictably sized and shaped specimen 516. To further encourage tearing at this point 514, the probe surface exterior to the concavity may optionally be insulated so as to not freeze adjacent tissue. This option is shown in FIG. 5D, described in more detail below. In an additional option which may be used together with the insulation option and/or may be used independently, a portion of the probe tip exterior to the concavity may be coated with a hydrophobic material to discourage adhesions of tissue to probe outside of the tearing lines shown in the figure. In an embodiment of the invention, the hollow cavity is symmetrical around a longitudinal axis of the cryoprobe.

In an exemplary embodiment of the invention, an optional thermal sensor 520 is provided to provide temperature feedback to a controller 522, where the controller 522 is programmed to change the heating and/or cooling and the degree of heating and/or cooling to the canula. The placement of the sensor in FIG. 5B is exemplary and not limiting. It should be understood that a temperature controller, like controller 522, could be used in any of the embodiments described herein which provide heating and/or cooling to at least a portion of the endoscopic cryoprobe system, for example the embodiments described in FIGS. 4A-4B.

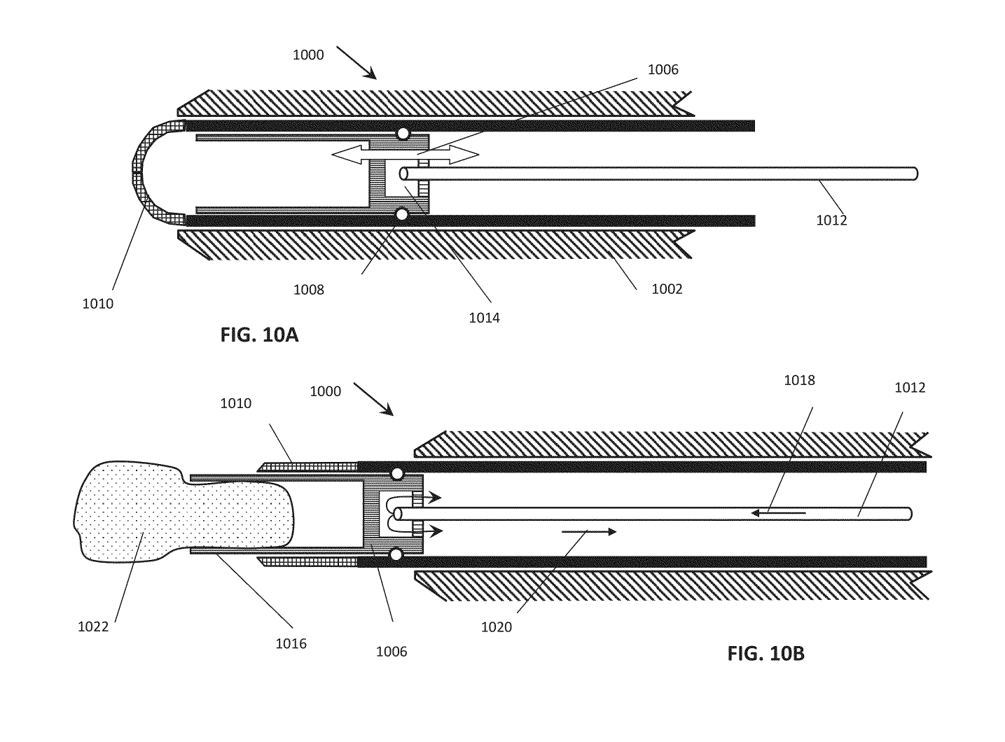

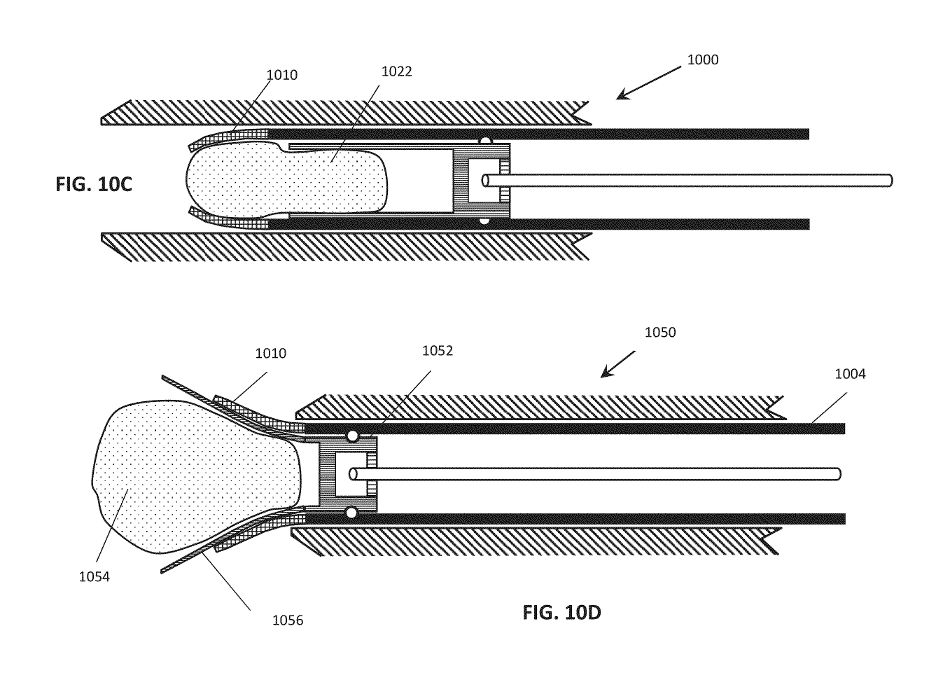

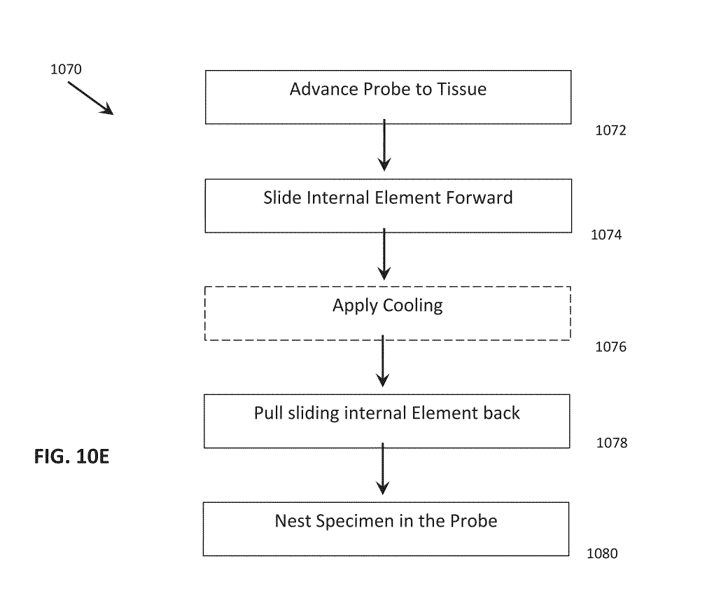

Pushing specially shaped cryotips 510, 540, 550 such as those shown in FIGS. 5B, 5C and 5D into most body tissues 512, 542, 552 will cause the tissues to be at least partially pushed into the hollow cavity, and once the tissue is frozen and the probe retracted, the start and end of tearing lines 514, 544, 554 (‘lines’ in these two-dimensional drawings, in realty disconnecting surfaces) in the tissue will tend to be at the (optionally sharp) edges 518, 548, 558 of the cavity. Indeed, in some embodiments, edges of the cavity may be narrow surfaces with sharp forward edges able to cut tissues when advance. In some embodiments these edges may be cooled not be direct contact with expanding gas but rather by conduction with surfaces which are in direct contact with expanding gas, as shown in FIG. 10B and discussed below.

Cooling the tissue inserted in the cavity and pulling the cooled probe away from the body tissue 512, 542, 552 will tend to make specimens 516, 546, 556 whose size is at least partially determined by the size and shape of the cavity, as shown in the Figures, and will therefore be relatively predictable from sample to sample. A conical cavity like that shown in FIG. 5B may work better for dense tissue, and a cylindrical hollow like that shown in FIG. 5C may work better in less dense tissue. These particular shapes are exemplary and not limiting, other forms of hollow cavity are also contemplated in the invention.

FIG. 5D shows a probe tip 550 that is cooled so that some of its surfaces are below freezing temperatures and others of its surfaces 558 remain above freezing temperature, for example to make the size and shape of tissue adhering to the probe more predictable. Various designs for the internal architecture of the cooling system of a probe can be used to cause some parts of the surface to cool more than others. Cooling gas can be directed by a nozzle to one part of a surface rather than another, in an embodiment of the invention. In some embodiments of the invention, a material 562 with low thermal conductivity is used to insulate parts of the surface of the probe from the influence of the probe cooling system, so that un-insulated parts will cool enough to cause tissue to adhere, while insulated surfaces 562 will not cool enough to cause tissue to adhere. In some embodiments of the invention, at least a portion of the cryotip 550 is covered with a hydrophilic material 560. An example of these techniques is shown in combination in FIG. 5D, where one part of a probe surface is directly exposed to a jet of cold gas, and another part of the probe surface is insulated by an insulating layer between it and the cold gas and where the tip is covered with a hydrophobic material to stimulate specimen adhesion. It should be understood that these configurations can exist separately or in any combination.

In some embodiments of the invention, a specially shaped sharp cryotip is used for penetrating the bronchus or tracheal wall so cytological samples (as is done with WANG needle or EBUS (Endobronchial Ultrasound Transbronchial Needle Aspiration (EBUS-TBNA)) can be retrieved in addition to samples for tissue analysis. In some embodiments of the invention, a blunt tip is used to decrease the likelihood of penetrating ling tissue and causing pneumothorax. It should be understood that many of the embodiments described herein could be employed with a blunt or sharp tipped cryoprobe.

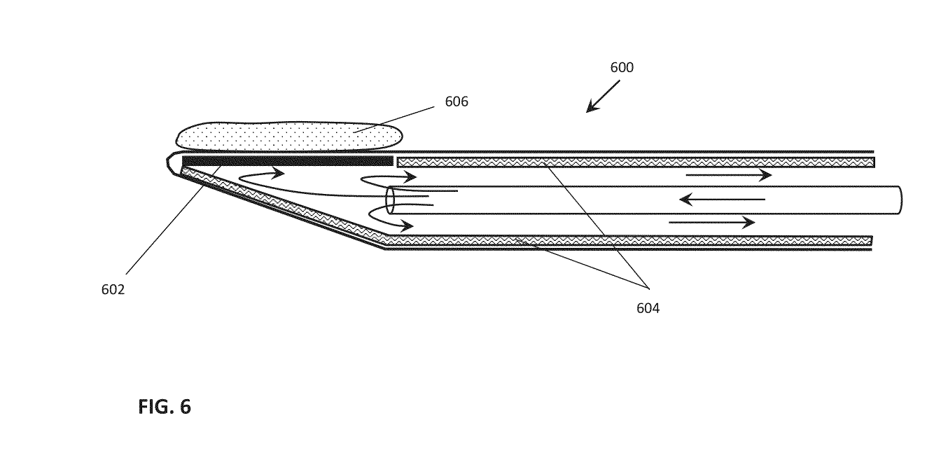

FIG. 6 is a schematic view of a side harvesting endoscopic cryoprobe 600, in accordance with an exemplary embodiment of the invention. FIG. 6 shows a design for a probe 600 which has one or more sides or surfaces 602 designed to cause adhesion of a biopsy sample/specimen 606, and other sides or surfaces 604 designed not to cause adhesion of body tissue to the surfaces 604. FIG. 6 shows a “side sampler” biopsy sampling device, with a probe tip made with a structure combining high thermal conductivity materials (a surface 602 designed to cause adhesion) and low thermal conductivity materials (a surface 604 designed not to cause adhesion). Cooling power in the form of recently expanded cryogenic gas is directed towards the surface 602 comprising a high thermal conductivity material, the high thermal conductive nature of the surface 602 causes the surface to cool as a result, thereby freezing the specimen 606 to it. The surfaces 604 protected from excessive cooling because they are made of relatively low thermal conductivity material do not cause body tissue adhesion because they are not cold enough to cause freezing, in an embodiment of the invention.

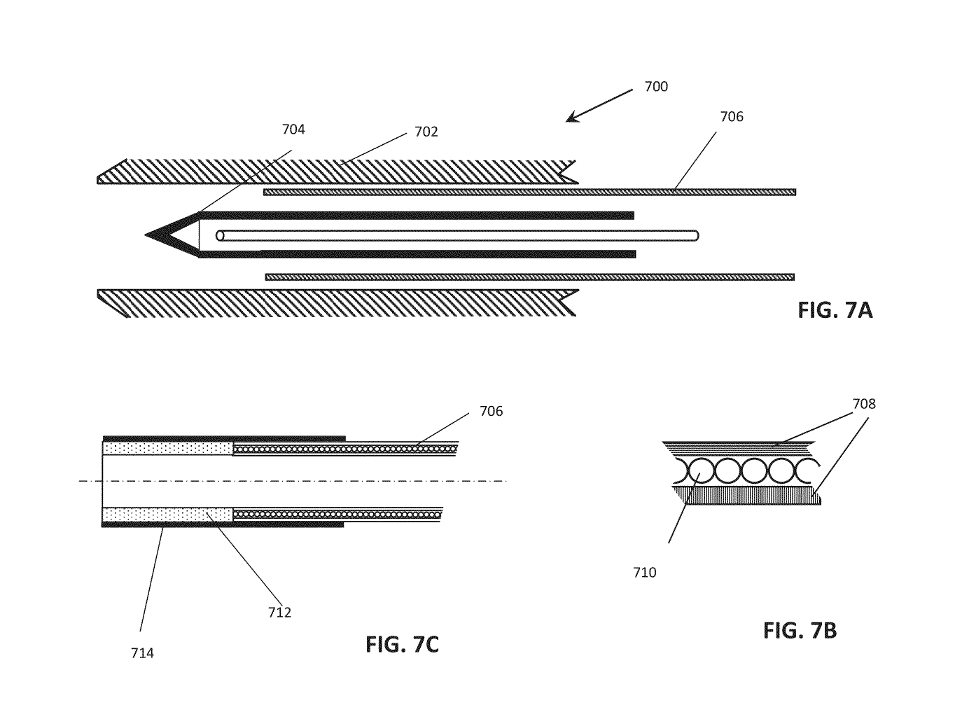

FIG. 7A is a schematic view of an endoscopic cryoprobe 700 with a kink resistant canula 706, in accordance with an exemplary embodiment of the invention. FIGS. 7A-7C describe a composite canula 706 which is kink resistant, has a small radius of curvature, and/or uses low-friction materials which make it easier to maneuver the canula 706 within the endoscope instrument channel 702. Regular plastics do not provide small radius of curvature and/or may be subject to severe flattening and kinking, thereby making it difficult, and even in some cases impossible, to insert and remove the canula 706 in the instrument channel 702 and/or to insert and remove the cryoprobe 704 from the canula 706. Consequently, in some embodiments we use composite materials and structures, such as braided or coiled structures 710, for example as shown in FIG. 7B, optionally coated on one or more sides with low friction plastics 708 (Polyimide, Nylon, Polyurethane, PTFE, etc). These composite structures can be bent in the small radii associated with the typical maneuvering of endoscopes, without kinking. In some embodiments of the invention, a hydrophilic coating that reduces dramatically the friction. Hydrophilic coating may be applied both on the outer surface of the cryoprobe and/or the inner and/or outer surface of the canula to achieve low friction movement of the canula and the cryoprobe relative to each other and to the endoscope.

FIG. 7C is a more detailed view of an endoscopic cryoprobe with a kink resistant canula and a transparent tip, in accordance with an exemplary embodiment of the invention. FIG. 7C describes optional transparent tubing 712 at the canula tip, optionally held in place via the use of a transparent ‘shrink tube’ 714. This transparent canula tip nests the gathered specimen (and cryoprobe) inside and provides better visualization during work.

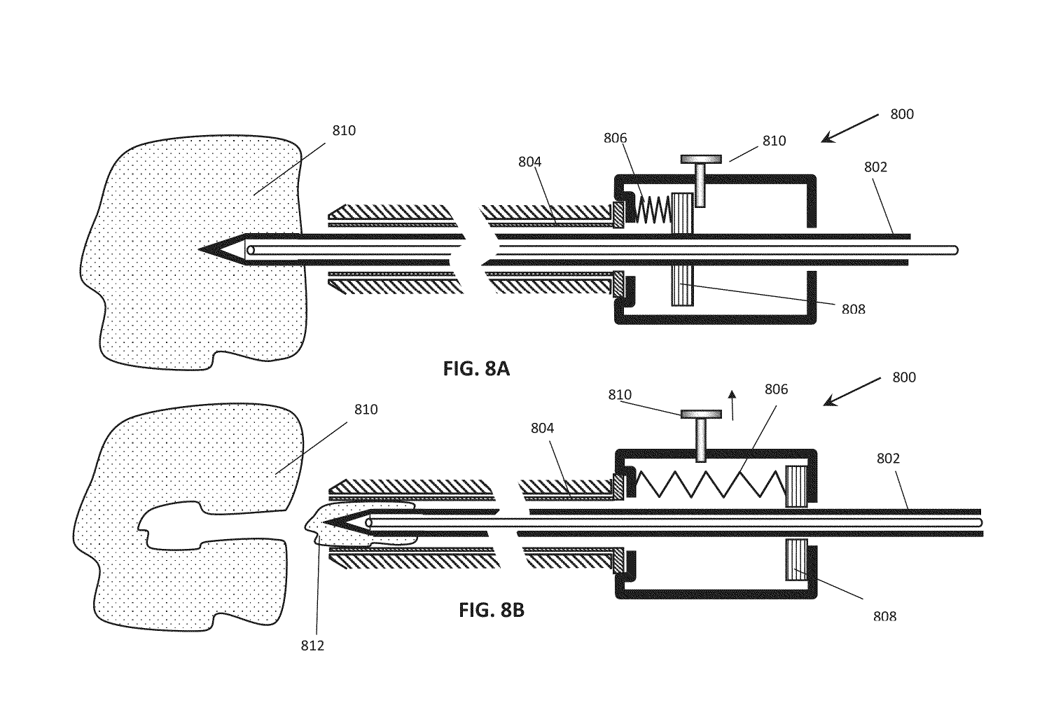

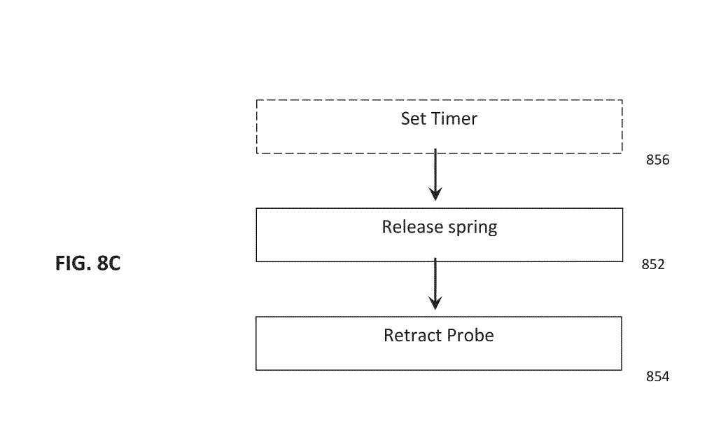

FIGS. 8A-8B are schematic views of an automatic tearing mechanism 800, in accordance with an exemplary embodiment of the invention. In some current methods of acquiring biopsy specimens, immediately after about 2-5 seconds of cooling (exact length of time depending on gas and flow) the physician pulls back on the cryoprobe to tear the specimen tissue from the bulk tissue. One cause of undesirable, varying specimen size is the difference between tearing force and speed from one tissue sampling to another and/or from one physician to another.

FIGS. 8A-8B describe an at least partially automatic tearing mechanism 800 for reducing size variability of the specimen 812 (sample to sample variability) and FIG. 8C is a flowchart 850 describing a method of using the mechanism 800 of FIGS. 8A-8B. A spring-loaded mechanism with one subassembly 808 attached to the cryoprobe 802 and the other subassembly attached to the endoscope 814 is provided, in an embodiment of the invention. As seen in FIG. 8B, pulling the knob 810 up releases (852) a compressed spring 806, which causes the cryoprobe 802 to retract (854) from the tissue 810 it has cooled, tearing the tissue in a highly repeatable manner. Optional features include a spring-based or electronic timer (856) to provide standardized timing for the cooling process and/or for any delays between cooling and pulling, so as to further standardize the tissue harvesting process and produce reliably similar results. In some embodiments of the invention, for convenience, a lever arrangement may be provided so as to provide a control to be pushed, rather than pulled, to initiate the freezing and/or the release process.

FIG. 9A is a schematic view of an endoscopic cryoprobe system 900 with a coolant source 910, in accordance with an exemplary embodiment of the invention. In some embodiments of the invention, the coolant source 910 is handheld and/or hand controlled by the attending physician, for example CryoAlfa handheld devices from Cryoswiss GmbH of Basel, Switzerland. In an embodiment of the invention, a valve 908 is used to control the volume of coolant introduced into the cryoprobe 902, which is itself inserted into a canula 912. In an embodiment of the invention, a lever 906 is used to control the valve 908 and/or the flow rate of the coolant into the cryoprobe 902.

In some embodiments of the invention, the system 900 may include a timer for applying the coolant gas during a pre-specified time period. The system 900 may utilize different cryotips, such as those described in this disclosure. The system may include specimen tearing means as are described in this disclosure. The system uses different types of canulas and/or coated canulas as described herein, in some embodiments of the invention. In an embodiment of the invention, the system uses different methods to pull out the specimen, for example using one of the methods described herein. In an embodiment of the invention, the valve 908 includes a knob with several positions allowing different flow rates and thus providing a control means with discrete settings to the cooling power.

The system optionally includes an internal temperature sensor and/or temperature controller, as described with respect to FIG. 5B. In an embodiment of the invention, coolant source 910 is filled with a Joule-Thomson gas such as N2O, CO2, Krypton, N2, Argon, and/or optionally Helium (for heating). An example of a coolant source is sold by H&O Equipments, Inc. in Mt. Pleasant, S.C., U.S.A.

The flexible cryoprobe optionally includes a heat exchanging configuration. The system optionally includes a gas filter of about 1-50 micron pores. Optionally, the filter has 1-10 micron pores. The filter is optionally a part of the coolant source or part of the cryoprobe. The cryoprobe and/or the gas cylinder are provided with quick replacement means, in some embodiments of the invention. The system is activated manually in some embodiments of the invention, or at least partially automatically, for example by a valve connected to a timer, or automatically by a valve connected to a controller. In an embodiment of the invention, the controller responds with feedback control based on information received from a sensor. Optionally, temperature control is rendered to any of the components of the endoscopic system, for example the cryoprobe, the canula or even the endoscope tube itself. Exemplary hand-held coolant sources with a small gas cartridge are disclosed in U.S. Pat. Nos. 7,150,743, 8,066,697, 6,706,037, 7,407,501, 7,402,160, 6,905,492, 6,905,492 by Zvuloni et al., the disclosures of which are incorporated herein by reference.

FIG. 9C is a cross-sectional view of the system 900 of FIGS. 9A-9B, in accordance with an exemplary embodiment of the invention. In an embodiment of the invention, the endoscope tube 914 includes an instrument channel 916, with a canula 912 and a cryoprobe 902 disposed therein, and optionally an imaging channel 918 and/or an illuminating channel 920.

FIG. 10A is a schematic view of an endoscopic cryoprobe system 1000 with a sliding internal element 1006, in accordance with an exemplary embodiment of the invention. As shown in FIG. 10A, a cryoprobe 1004 is provided with a sliding internal element 1006 which slides forward and backward within the cryoprobe tip. In an embodiment of the invention, this design allows a user to advance the sliding element 1006 into tissue, cool it to harvest the specimen 1022, and then retract it into the probe 1004 body, ‘nesting’ it inside the probe 1004 so that the specimen 1022 is protected, and so that the specimen 1022 is removed, without removal of the endoscope 1002, by retracting the probe 1004 and/or by retracting a canula containing the probe.

In an embodiment of the invention, maneuvering of the sliding element 1006 is accomplished by moving the high pressure gas tube 1012, which is firmly connected to the sliding element 1006. Alternatively, the sliding element 1006 is maneuvered via an independent wire, or by other means. A sealing element 1008, such as an o-ring, between the cryoprobe 1004 and the sliding element 1006 prevents the low pressure returning gas 1020, shown in FIG. 10B, from flowing out of the tip. In an embodiment of the invention, flexible ‘wings’ 1010 at the probe tip, optionally having a hemispherical or similar structure, allow smooth movement of the cryoprobe 1004 forward inside the endoscope instrument channel.

In some embodiments of the invention, a canula is provided with the flexible wings at the distal end and a sliding element within, where the sliding element functions as the cryoprobe in the endoscopic system.

FIGS. 10B-10C are schematic views showing a sampling procedure using the endoscopic cryoprobe system 1000 of FIG. 10A, in accordance with an exemplary embodiment of the invention. In use, once the endoscope 1002 is in place the cryoprobe 1004 may be advanced (1072), shown in the flowchart 1070 in FIG. 10E, beyond the endoscope channel end to penetrate nearby tissue. At this point, the sliding internal element 1006 slides (1074) forward through the ‘wings’ 1010 and into the tissue. As shown in FIG. 10B, cooling is optionally applied (1076) and the cooling elements 1016 (optionally blade-like, optionally cylindrical, optionally having other forms) are cooled down to sub-zero temperatures, causing them to adhere to tissue. The cooling elements 1016 are optionally made from highly conductive material such as aluminum.

As shown in FIG. 10C, the sliding element, after cooling and with adhering tissue, is pulled (1078) backward, thereby harvesting the tissue by tearing the tissue from its site, and nesting (1080) a specimen 1022 inside the cryoprobe 1004. The harvested tissue is further protected by the flexible ‘wings’ 1010 which may close once the sliding element 1006 is retracted from the tissue.

FIG. 10D is a schematic view of an endoscopic cryoprobe system 1050 with a sliding and expanding internal element 1052, in accordance with an exemplary embodiment of the invention. In an embodiment of the invention, not only the ‘wings’ 1010 but also the cooling elements 1016 are made flexible and/or are shaped or pre-bent to open when sliding forward, into a form which opens wider than the total diameter of the canula (or wider than the diameter of the cryoprobe and/or wider than the diameter of the endoscope instrument channel). In an embodiment of the invention, tissue samples/specimens which are initially larger than the cryoprobe 1004 diameter can be harvested. This is useful because particularly when seeking tissue samples for biopsies of lung tissue, it is often difficult to harvest a sufficient amount of tissue.

Pre-bent springy metal shapes and/or shape-memory alloys such as Nitinol can be used to cause the cooling elements to assume this larger diameter, expanded shape. Using the embodiment of FIG. 10D a user can advance the slidable cooling elements 1056 into a body of tissue larger in diameter than the size of the probe 1004 from which the elements advance, optionally using a sharp forward edge to cut rather than push the tissue, then cool the elements 1056 to freeze the tissue, pull back on the probe 1004 to disconnect the frozen tissue from the body, then gradually allow partial melting of parts of the frozen specimen 1054 somewhat distanced, for example roughly 0.5-2 mm, from the cooling elements 1056 in coordination with withdrawing the specimen 1054 and cooling elements 1056 into the probe 1004 body. The effect will be a gentle compression of the harvested specimen 1054, but since in the case of lung tissue the tissue is not dense and largely penetrated with air spaces, such gentle compression in controlled amounts may accomplish the harvesting of a relatively large amount of tissue without destroying or excessively distorting the cellular and intra-cellular structures of the tissue.

In an embodiment of the invention, the ‘wings’ 1010 shown in the figure and/or the external portions of the cooling elements 1056 may comprise or be coated with material which is hydrophobic and/or thermally insulating so that the forward-extended cooling elements 1056 will cool tissue within the cooling element structure to facilitate harvesting, yet will have less tendency to adhere to tissues outside the structure, thereby tending to create standardized sample sizes.

In some embodiments of the invention, the sliding internal element 1006, 1052 of FIGS. 10A-10D include a Joule-Thomson expansion chamber and further include relatively long and/or narrow blades and/or surfaces which are somewhat distanced, for example roughly 2-8 mm, from the Joule-Thomson expansion chamber and which project forward into body tissue when the sliding element is advanced. These blades or surfaces (optionally flat, optionally forming a cylinder or other closed surface) optionally have sharp front edges to facilitate penetration into tissue. These elements extend forward of the Joule-Thomson expansion chamber and, in an embodiment of the invention, are cooled by thermal conduction between the chamber and the ‘blades’ or other advancing structures. In other words, in an exemplary embodiment of the invention, the cooling elements used to cool tissue is distanced from the gas expansion chamber used to cool the cooling elements. The cooling elements optionally extend some distance away from the chamber, for example roughly 2-8 mm, and may be connected to the chamber by highly thermally conductive material. In an embodiment of the invention, the cooling elements include thermal insulation along some of their length and/or on some of their surfaces, so as to distance the tissue cooling from the position of the gas expansion. Additionally, alternatively and/or optionally, the cooling elements may include thermal insulation along some of their surfaces so as to freeze and/or adhere tissue on some surfaces (e.g. inside a cavity) and not on other surfaces (e.g. outside the cavity), for example as discussed elsewhere herein.

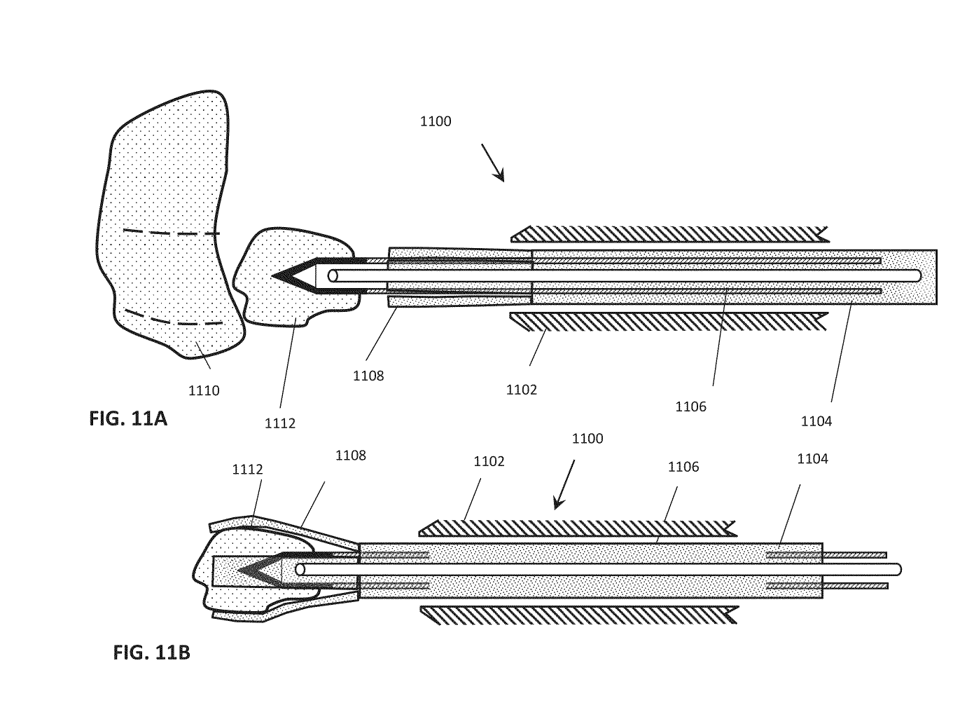

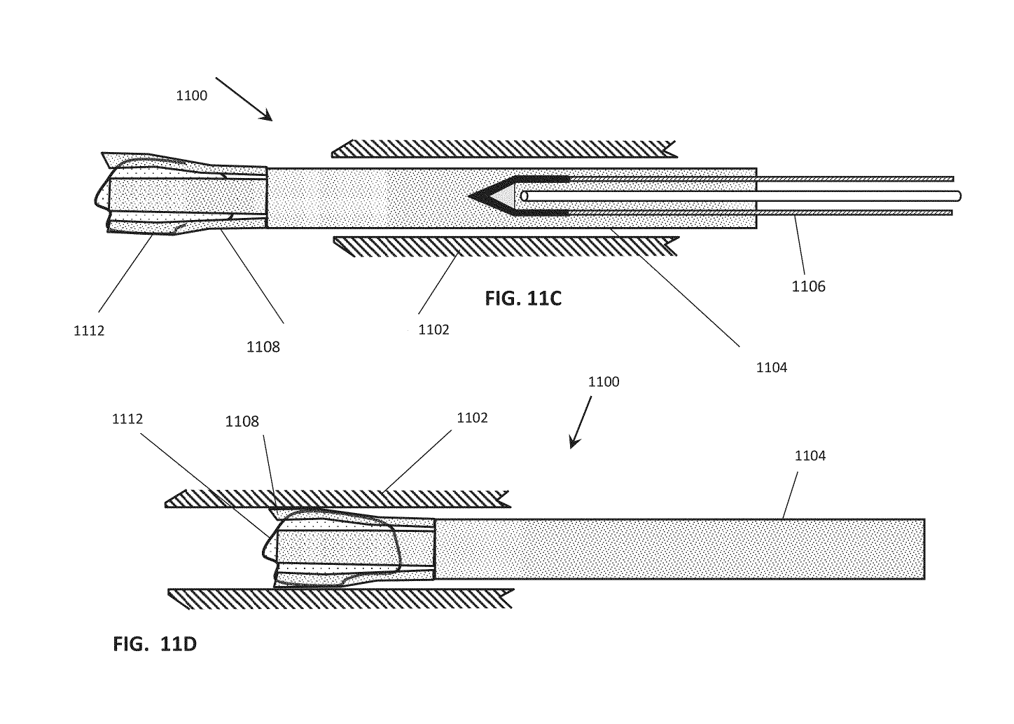

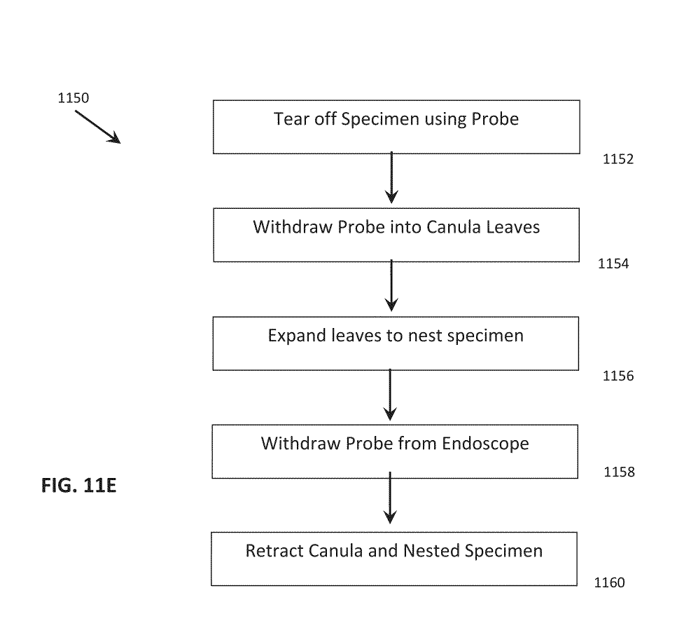

FIGS. 11A-11E are a graphical and flowchart 1150 depiction of a sampling procedure using a endoscopic cryoprobe system 1100 with an expanding leaf canula 1104 including an expanding leaf tip 1108, in accordance with an exemplary embodiment of the invention. In an embodiment of the invention, the procedure for tearing (1152) off a specimen 1112 from body tissue 1110 is substantially the same as described elsewhere herein, however, in some embodiments of the invention, the probe 1106 is withdrawn (1154) into the canula 1104 such that the expandable, flexible leaves at the tip 1108 of the canula 1104 expand (1156) according to the size of the specimen 1112, as shown in FIG. 11B. In an embodiment of the invention, the nested specimen 1112 remains in the leaves of the canula 1104 while the cryoprobe 1106 is withdrawn (1158), as shown in FIG. 11C. FIG. 11D shows the retraction (1160) of the canula 1104 with the nested specimen 1112 which, in some embodiments of the invention, is compressed to fit within the endoscope during withdrawal depending on the size of the specimen 1112.

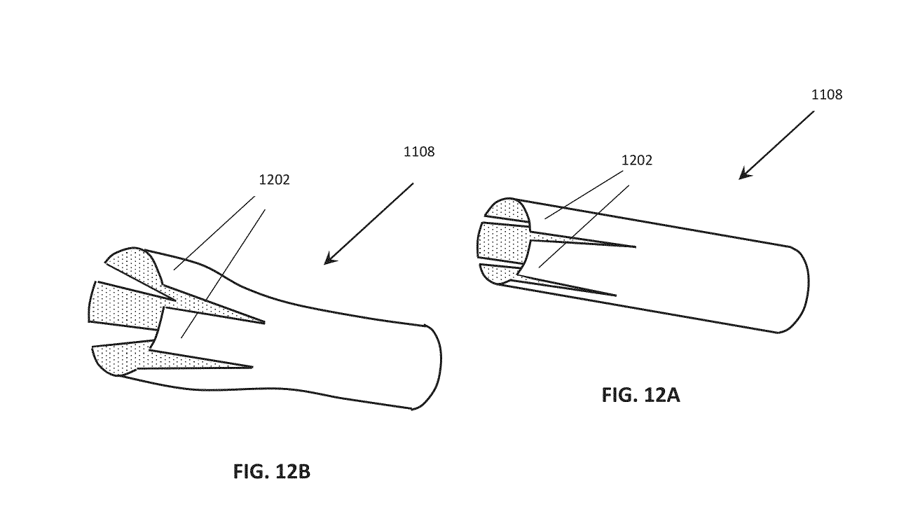

FIGS. 12A-12B are a perspective view of the tip 1108 of the expanding leaf canula of FIGS. 11A-11D, showing the leaves 1202 in more detail, in accordance with an exemplary embodiment of the invention.

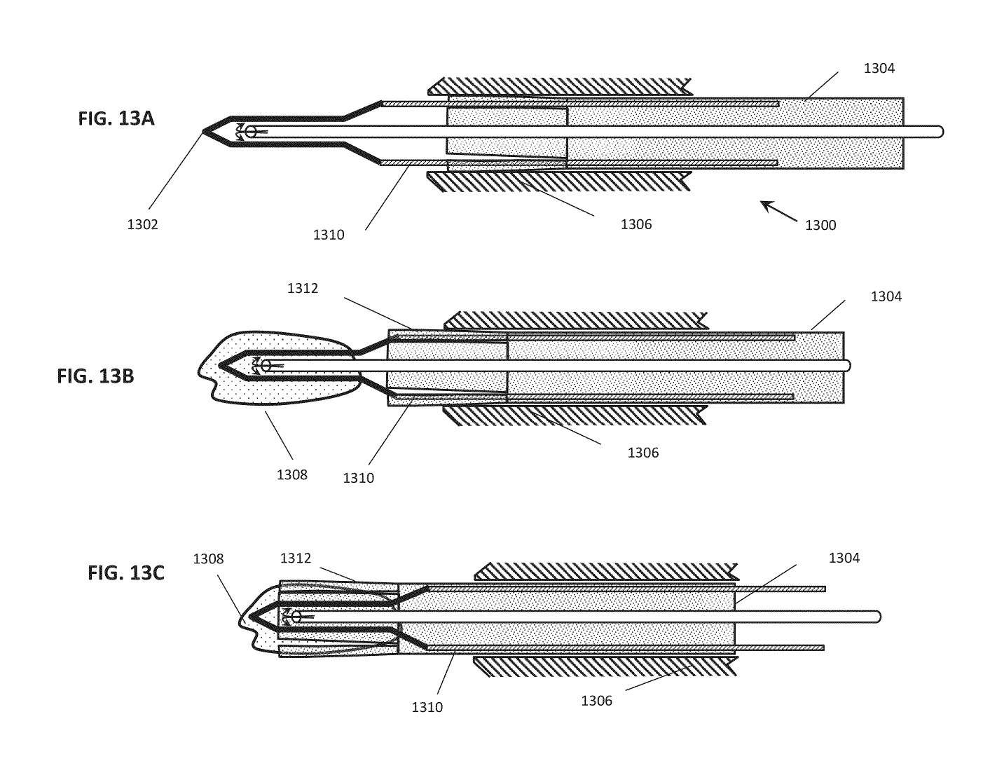

FIG. 13A-13C are a schematic view of an endoscopic cryoprobe system 1300 with a reduced profile cryoprobe tip 1302, in accordance with an exemplary embodiment of the invention. In an embodiment of the invention, the tip 1302 of the cryoprobe 1310 is reduced in profile (e.g. narrower in diameter) to provide for more room, in relation to current cryoprobe tips, within a canula 1304 or an endoscope instrument channel 1306 for a retrieved specimen 1308, shown in FIG. 13B.

FIG. 13C shows how a combination of features of the endoscope cryoprobe system 1300 work together to enhance the size of the specimen 1308 that can be collected, in an embodiment of the invention. In some embodiments of the invention, the cryoprobe tip 1302 is provided with expanding (in a radial fashion) and/or flexible leaves 1312, such as those described with respect to FIGS. 11A-11E. In concert, the smaller diameter cryoprobe tip 1302, described with respect to FIGS. 13A-13B, and the expanding canula 1304 permit a larger specimen 1308 to be collected than was previously possible. In an embodiment of the invention, the probe is pulled back after tearing off the specimen and is nested within the expanding leaves 1312 of the canula. In an embodiment of the invention, the canula and the cryoprobe, along with the specimen, are withdrawn from the endoscope. Optionally, temperature control and/or composite/layered construction permit the withdrawal of the specimen with either the probe or the canula alone, such as described elsewhere herein, depending on the desires or needs of the physician and/or patient.

It should be understood that while certain new shapes for cryoprobe tips are described in the specification, for example in FIGS. 5B-5D, 6, and 10A-10D, some embodiments of the invention are applicable to the commonly available cryoprobe designs today. For example, the expanding leaf canula design of FIGS. 11A-11D, 12A-12B and 13A-13C could be used with a current cryoprobe; the automatic tearing mechanism of FIGS. 8A-8B could also be used with currently available cryoprobes; certain of the composite or layered material canulas described herein could also be used with currently available cryoprobes; and, certain of the temperature controlled canulas described herein could also be used with currently available cryoprobes. It is expected that during the life of a patent maturing from this application many relevant endoscopic tools will be developed and the scope of the term endoscope is intended to include all such new technologies a priori.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. An endoscopic cryoprobe system, comprising:
a low friction canula slidable within an instrument channel of an endoscope tube; and,
a low friction cryoprobe slidable within the canula and provided with a tissue specimen acquiring tip at the distal end of the cryoprobe, where the canula is sized and shaped to nest the cryoprobe and the specimen within for protection during retraction.

2. A system according to claim 1, where the specimen is retained by at least one of the canula or cryoprobe during retraction by at least one of a compression fit, frozen adhesion, adhesion to a hydrophilic material or by being speared by the tip.

3. A system according to claim 1, with the tip comprising sharpened edges at the leading edge of the tip and a hollow cavity extending from the leading edge towards the proximal end of the tip.

4. A system according to claim 3, where the hollow cavity is symmetrical around a longitudinal axis of the cryoprobe.

5. A system according to claim 3, where the hollow cavity is shaped like a cone, with the base of the cone at the leading edge.

6. A system according to claim 3, where the hollow cavity is shaped like a cylinder.

7. A system according to claim 1, with the canula comprising temperature control circuitry including at least one of switching polarity conductive wiring or resistive heating conductive wiring, and capable of providing at least one of heating or cooling to selected surfaces of the canula.

8. A system according to claim 7, where selected surfaces are at least a portion of at least one an inner surface of the canula, an outer surface of the canula, an outer surface of the cryoprobe, or an inner surface of the cryoprobe.

9-12. (canceled)

13. A system according to claim 1, where at least a portion of at least one of the cryoprobe or the canula is coated with a hydrophobic coating to enhance adhesion.

14. A system according to claim 1, where at least a portion of at least one of the cryoprobe or the canula is coated with a hydrophilic coating to reduce adhesion.

15. A system according to claim 3, where an inner surface of the cavity is cooled to enhance adhesion of the specimen thereto.

16. A system according to claim 1, wherein said canula is temperature controlled and has at least one heated or cooled surface; and wherein said system further comprising a sensor and a temperature controller, wherein said controller is programmed to change the heating or cooling and the degree of heating or cooling to said canula in response to feedback from said sensor.

17. A system according to claim 16, where the at least one heated or cooled surface is at least one of an inside surface of the canula or outside surface of the canula.

18-20. (canceled)

21. A system according to claim 1, wherein said tip comprises an expanding portion.

22-23. (canceled)

24. A method for performing an endoscopic cryoprobe biopsy, comprising:
cooling a tip of a cryoprobe in contact with body tissue, causing the tissue to be sampled to adhere to the tip through freezing;
pulling the cryoprobe tip along with a torn off tissue specimen back into a canula;
pulling the canula out of an endoscope tube inserted into the body lumen with the cryoprobe and the specimen nested within for protection; and,
removing the specimen from the canula for storage.

25. A method according to claim 24, where the tip is a shaped tip and further comprising pushing the shaped tip with a hollow cavity into the tissue to be sampled, thereby forcing and adhering the tissue into the cavity, prior to pulling the cryoprobe tip back into the canula.

26. A method according to claim 24, further comprising automatically tearing off the tissue specimen using an automatic tearing mechanism activated by a spring loaded mechanism to increase consistency of the sampling.

27. (canceled)

28. A method according to claim 26, further comprising using a timer to set the timing of at least one of the retraction of the automatic tearing mechanism or the application of temperature control to the cryoprobe.

29. (canceled)

30. A method according to claim 24, where the specimen is pulled back into an expandable cryoprobe tip thereby retrieving a specimen larger in diameter than at least one of the cryoprobe or canula.

31. (canceled)

32. A method according to claim 24, further comprising encouraging adhesion of the specimen to at least a portion of the system by performing at least one of cooling the portion of the system or coating the portion with an adhesion facilitating coating.

33-36. (canceled)
